(19) United States
(12) Patent Application Publication
(10) Pub. No.: US 2004/0167629 A1
(43) Pub. Date: Aug. 26, 2004

(54) Modular shoulder prosthesis

(76) Inventors: Perry A. Geremakis, Manalapan, NJ (US); Roy Hori, Bellevue, WA (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/763,574

(22) Filed: Jan. 22, 2004

Related U.S. Application Data
(60) Provisional application No. 60/444,505, filed on Feb. 3, 2003.

(51) Int. Cl. A61F 2/30; A61F 2/40

(52) U.S. Cl. 623/19.14; 623/22.42; 623/23.42; 623/23.47

(57) ABSTRACT

The present invention relates to a modular joint prosthesis for replacement of an affected joints and, particularly, an affected shoulder joint. The joint prosthesis of the current invention comprises a head having multiple components which can be assembled with each other in-situ during the course of a surgery. Consequently, the patient implanted with the modular joint prosthesis would experience minimized incision, accelerated recovery, and decreased scarring.

### CROSS REFERENCE TO RELATED APPLICATIONS

This application claims priority from provisional application Ser. No. 60/444,505, filed on Feb. 3, 2003.

### BACKGROUND

1. Field of the Invention

This invention relates to devices that would be implanted in joints to allow restoration of function, as well as relief of pain, to an effected joint. Specifically, this invention relates to a shoulder prosthesis for use in replacement of a damaged or diseased shoulder.

2. Description of Related Art

As the population ages, while simultaneously tries to maintain an active lifestyle, injuries and ailments as a result of such lifestyles will increase. These include overuse, deterioration/arthritis, as well as trauma. Injuries to soft tissues, such as muscles, ligaments, and tendons are often treated through a less invasive, or even a minimally invasive approach, whereby the surgeon minimizes the incision to the effected site. The advantages of a smaller incision include faster recovery time and less scarring. When injuries affect the major joints of the body, such as the hip, knee, and shoulder, surgeons can replace these damaged joints with artificial ones, for the purposes of relieving pain as well as restoring function. The replacement of these joints is an invasive procedure, usually with a large incision, which results in slow recovery times and scarring. It would be advantageous to provide a system for joint replacement, specifically shoulder replacement, that would be less invasive, as a result of a minimized incision, to accelerate recovery and decrease scarring.

The shoulder joint is the joint in the body that has the greatest and most diverse ranges of motion. As shown in FIG. 15, the shoulder joint is composed of the head of the humerus 22 articulating against the glenoid portion 2 of the scapula. In addition to the humeral/glenoid joint, there is the acromioclavicular joint located superior to the humeral/glenoid joint. Shoulder replacement prostheses focus on replacing the humeral/glenoid joint. A variety of muscles, ligament, and tendons serve to constrain the joint, represented by 23a and 23b. Additional pictorial descriptions are shown in FIGS. 16 and 17. In part because of the complex motions that this joint affords, replacement of this joint with a prosthetic shoulder has lagged that of other joints such as the hip and the knee. However, recent designs have increased the success rate of shoulder joint replacement, and have therefore resulted in a greater acceptance of this type of surgery as a means for surgeons to address problems such as disabling overuse, arthritis, and trauma. Shoulder replacement systems are typically composed of two parts: a humeral component and a glenoid piece. The distal portion of the humeral component is a stem which is seated inside the medulary canal of the humerus. The proximal portion of the humeral component is a section of a generally spherical head. This head serves to replace the head of the humerus, which is resected during the replacement surgery. The glenoid component is the piece that the humeral head articulates against. The glenoid component is typically either a single piece of medical grade plastic, or a two-part assembly consisting of a metal backing with a plastic insert. The glenoid component is fixed, usually with the aid of bone cement, or if done without bone cement, with the aid of bone screws, to an appropriately contoured portion of the glenoid, and the smooth plastic side opposite the fixed side, is that which the humeral head articulates against.

Early versions of shoulder prostheses consisted of a humeral component that was a single unitary structure. These were gradually replaced by structures that offered the surgeon a two piece construct consisting of a stem and a head. Typically the head is press-fit through a taper-lock mechanism to the stem for a rigid attachment. This modularity offers the surgeon more choices in terms of customizing sizes, which allows the surgeon to better replicate the anatomy and function of the effected shoulder joint. Additionally, modularity requires less inventory be kept on hand, and allows a cost savings, from the perspective of hospitals. However, since the head component is still a unitary structure, the incision through which the implant is placed has not decreased. If the head were to be composed of several pieces, and assembled in-situ, then the patient could reap the benefit of a smaller incision and faster recovery time.

### SUMMARY OF THE INVENTION

The present invention is a joint prosthesis that has distal end composed of an elongated stem portion adapted to fit within the medulary canal of a long bone, and a proximal end, which has a substantially different shape from the distal end, and is adapted to mate with at least one head component. Alternatively, such distal end of long bone component can be adapted to mate with an attachment plate, such plate being adapted to mate with at least one head component. Additionally, a second component is offered to for the head component to articulate against.

The modular nature of the head component will allow the surgeon to make a smaller incision to implant the prosthesis, thereby increasing the recovery time of the patient. The modular head can be composed of two or more articulating pieces, which depending on their assembly, could allow the surgeon to perform the operation from one of several different surgical approaches, as will be described in more detail below. The preferred embodiment of such joint prosthesis is that of a shoulder joint, although it will be understood to those skilled in the art that the inventions described herein are applicable to other joints such as the hip, knee, digits, elbows, ankles, and intervertebral joints.

### BRIEF DESCRIPTION OF THE DRAWINGS

Preferred embodiment(s) of the present disclosure are described herein with reference to the drawings wherein:

FIG. 1 is an isometric view of a modular shoulder prosthesis in accordance with the principles of the present disclosure, with one modular head component assembled on a stem.

FIG. 2 is an isometric view of a modular shoulder prosthesis, with three modular head components assembled on the stem.

FIG. 3 is a partial bottom view of the assembled three modular head components of FIG. 2.

FIG. 4 is a partial isometric view of the modular shoulder prosthesis of FIG. 2, with the assembled modular head components further locked and leveled through a lock.

FIG. 5 is a partial side view of the modular shoulder prosthesis of FIG. 2, showing articulation against a glenoid component.

FIG. 6A is a top view schematically of a portion of the stem component of FIG. 2.

FIG. 6B is a front view schematically of the stem component of FIG. 2.

FIG. 6C is a cross-sectional view schematically taken across 6C-6C of FIG. 6B to illustrate a side view of the stem component.

FIG. 7A is a side view schematically of one modular head component of FIG. 2.

FIG. 7B is a front view schematically of the one modular head component of FIG. 2.

FIG. 7C is a top view schematically of the one modular head component of FIG. 2.

FIG. 7D is an isometric view schematically of the one modular head component of FIG. 2.

FIG. 8A is an isometric view schematically of a lock in accordance with the principles of the present disclosure.

FIG. 8B is another isometric view schematically of the lock of FIG. 8A.

FIG. 9 is a partial isometric view of an alternative modular shoulder prosthesis in accordance with the principles of the present invention, with one modular head component assembled on a platform on the stem.

FIG. 10 is a partial isometric view of the modular shoulder prosthesis of FIG. 9, with two modular head components assembled on the platform.

FIG. 11 is an isometric view of the modular shoulder prosthesis of FIG. 9, with three modular head components assembled and fixed by pin.

FIG. 12 is a partial isometric view of the modular shoulder prosthesis of FIG. 11.

FIG. 13A is a top view schematically of one modular head component of FIG. 9.

FIG. 13B is an isometric view schematically of the modular head component of FIG. 13A.

FIG. 13C is a side view schematically of the modular head component of FIG. 13A.

FIG. 13D is another side view schematically of the modular head component of FIG. 13A.

FIG. 14 is a isometric view schematically of the platform and stem of FIG. 9.

FIG. 15 is a schematic diagram showing a human shoulder.

FIG. 16 is an illustration of the bones of a human shoulder.

FIG. 17 is an illustration of the muscles, tendons, and ligaments of a human shoulder.

FIG. 18 is an isometric view of an alternative embodiment of the modular shoulder prosthesis in accordance with the principles of the present disclosure.

FIG. 19A is a cross-sectional view schematically taken across 19A-19A of FIG. 19B to illustrate a side view of the modular shoulder prosthesis.

FIG. 19B is a front view schematically of the modular shoulder prosthesis of FIG. 18.

FIG. 19C is a isometric view schematically of the modular shoulder prosthesis, showing the components used in this assembly.

FIG. 20A-20F are isometric views schematically of various embodiments of the platform that can be used in accordance with the principles of the present disclosure.

FIG. 21 is a partial isometric view of a height adjustable platform assembled on stem.

FIG. 22A is a cross-sectional view schematically taken across 22A-22A of FIG. 22C to illustrate a side view of the platform-stem assembly of FIG. 21.

FIG. 22B is an enlarged view schematically of the circled portion of FIG. 22A.

FIG. 22C is a front view schematically of the height adjustable platform assembled on stem of FIG. 21.

FIG. 23 is an isometric view of an alternative embodiment of the modular head components in accordance with the principles of the present disclosure.

FIG. 24A is a cross-sectional view schematically taken across 24A-24A of FIG. 24B to illustrate a bottom view of the modular head component of FIG. 23.

FIG. 24B is a side view schematically of the modular head component of FIG. 23.

FIG. 24C is a top view schematically of the modular head component of FIG. 23.

FIG. 24D is an isometric view schematically of the modular head component of FIG. 23.

FIG. 24E is a side view schematically of the modular head component of FIG. 23.

FIG. 25 is an isometric view of another alternative embodiment of the modular head components in accordance with the principles of the present disclosure, which is capable of being attached to a retaining ring.

FIG. 26A is a cross-sectional view schematically taken across 26A-26A of FIG. 26B to illustrate a side view of the modular head components of FIG. 25.

FIG. 26B is a side view schematically of the assembled modular head components of FIG. 25.

FIG. 26C is an isometric view schematically of the assembled modular head components of FIG. 25.

FIG. 26D is a bottom view schematically of the assembled modular head components of FIG. 25.

### DETAILED DESCRIPTION OF THE INVENTION

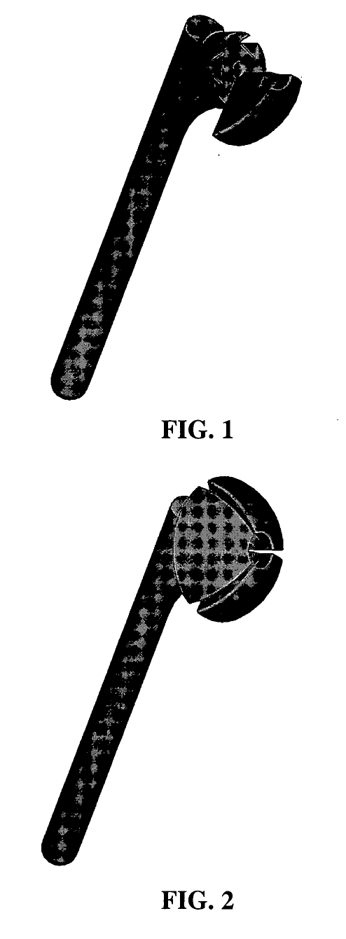

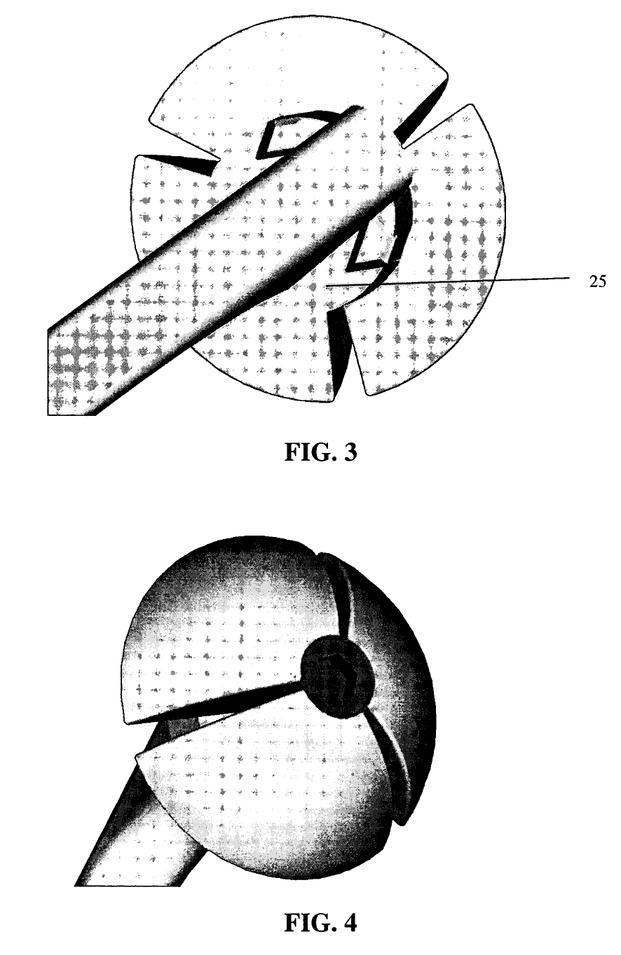

One embodiment of the invention is shown in FIGS. 1-8B, where the modular shoulder prosthesis is a multi-piece construct capable of being assembled in-situ. (Shoulder-1). Shoulder-1 is made from materials suitable for surgical use, and the assembled implant may be composed of multiple types of materials. For example, it is typical for articulating “ball and socket” prostheses to have a metal ball joint articulating against a polymer socket component. Materials include, but are not limited to, the following, and may be used alone or in combination as the designs dictate: stainless steels, cobalt chrome molibdinum alloys, titanitum, titanium alloys, ceramics, plastics. Enhanced fixation of the humeral component, and the stem portion which is implanted within the medulary canal in particular, may be achieved through surface roughenings or surface treatments, such as plasma spray coatings, porous bead coatings, bone cement pre-coatings, fiber metal mesh, blasting, etc. The components of Shoulder-1 may also be treated with an external coating to encourage enhanced fixation, such as through plasma spray coating, porous bead coatings, bone cement pre-coating, etc. Additionally, components may be made from a combination of appropriate materials so that the composite structure better approximates the mechanical properties of the host tissues and bones, such as a stem whose core is metal, yet has an outer shell made from a polymer or other material as appropriate to the design, thereby better matching the modulus of elasticity of the implant to the humerus bone.

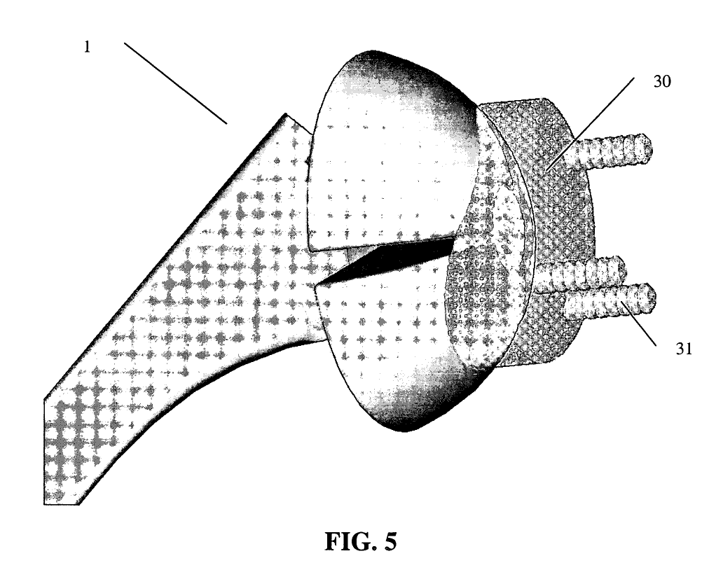

Once the initial exposure is performed, and the shoulder joint visualized, the surgeon will decide if only the humerus will be replaced or if both the humerus and the glenoid will be replaced. If both components are to be replaced, the surgeon will replace the glenoid component first with a glenoid implant 30, as shown in FIG. 5. Pegs 31 of the glenoid compenent are used to enhance fixation. These pegs 31 are typically inserted into holes drilled into the scapula and then filled with cement just prior to implanting the glenoid component 30. The pegs 31 may have grooves, or other such surface roughenings, to enhance fixation to the bone cement.

When the surgeon is ready to begin work on the humerus, the soft connective tissues from the humeral head will be seperated, so that the head can be resected. Although the head may be resected as a unitary structure, it is envisioned that it may be resected in pieces, thereby further reducing the incision and minimizing trauma to the surrounding soft tissues. A template or guide (not shown) would aid in this process, ensuring that the surgeon makes the appropriate cut in terms of angle and amount of humeral head resected. With the head resected, the surgeon then begins preparation of the medularly canal of the humerus. This preparation will involve reaming, rasping, broaching (medulary canal preparation instruments), or a combination thereof, performed using either manual or powered instruments. This canal preparation will ensure that an adequate fit results when the stem component of the implant is inserted. Typically, the reaming, rasping, and broaching is done sequentially, beginning with smaller sizes and increasing in dimension until the surgeon decides that the appropriate size has been reached. Also, it is common that the final size of the final medulary canal preparation instrument will serve as a template for the stem component. For implant systems that will use cement to enhance fixation, such final instrument is usually slightly larger than the implant size to be used. For implant systems that will rely on press-fit, such final instrument is usually slightly undersized in comparison to the final implant size. The use of bone cement, or other such grouting agents, may be used, or the stem could be press-fit into the prepared medulary canal.

Once the medulary canal of the humerus is prepared, the stem component is inserted. The stem component may have macro-surface roughenings, such as grooves, to better lock the stem into the canal, and into the cement if cement is being used. Such grooves could be vertical, or, a series of concentric rings encircling the long axis of the stem, at least one spiral groove going from the distal tip of the stem to some portion near the proximal end at which the head component will be attached. The stem is inserted so that underside 25 of platform 3 is resting against the resected surface of the humerus (not shown). This flat resected surface will allow the flushly seated platform to distribute compressive loads through the cortical bone of the humeral shaft. Once the stem has been implanted, the head components 10 can be assembled to the platform.

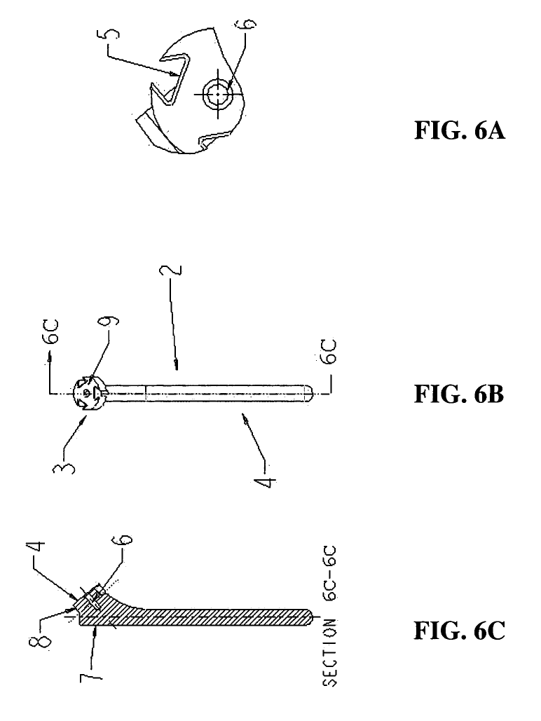

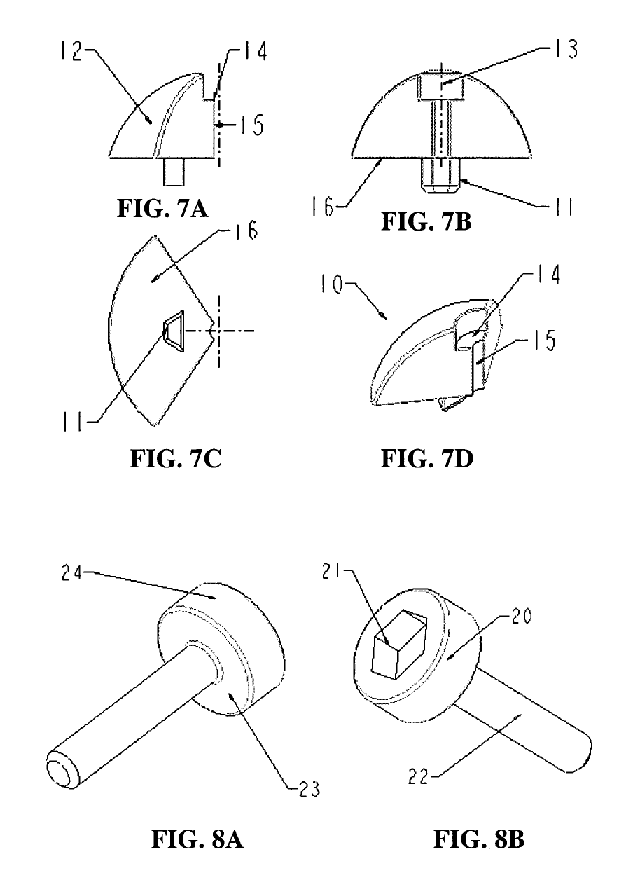

The modular humeral head is composed of at least two components, preferably three components, as shown in FIGS. 2-4. In the preferred embodiment, the head component 10 contains a peg 11, as shown in FIGS. 7A-7D, such peg is inserted into a respective slot 5 in the platform, as shown in FIG. 6A. Such slot is configured to receive such peg, and preferably, there is a taper lock engagement between the two so that the head components are fixedly engaged to the platform, as shown in FIG. 4. Additionally, the engagement between the slot and peg need not be a trapazoidial, but could be circular, triangular, square, or any other appropriate shape. The platform need not be limited to a circular shape, but could be oval, rectangular, or any other appropriately conforming shape. Alternatively, the engagement between slots 5 and pegs 11 need not be a locking engagement, but merely a snug fit; the components would be locked and leveled through a fastener 20 that engages and locks into the platform in 6 (via threading, press-fit, taper lock, adhesive, etc.). When surfaces 23 of fastener 20 engages all surfaces 14 of each of the head components 10, such fastener will serve to level the multiple head components and ensure that the resulting articulation is smooth. Once the head components have been secured, the underside 16 of the head components mate with the top surface 9 of the platform. Since the head components are assembled in-situ, the incision and subsequent disruption of nearby soft tissues is greatly reduced, thereby decreasing trauma to these tissues, and increasing the recovery time of the patient. Once the head components have been assemebled onto the platform, and fastened and leveled, this completes assembly of the humeral component.

Other embodiments of Shoulder-1 include having the head components dimensioned so that a gap exists between them, to allow for easier assembly. Additionally, such head components can approximate a circular or oval shape.

Another embodiment envisions the platform to be adjustable, so that the offset height can be varied. Such adjustability could be attained through multi-piece threaded mechanisms, whereby threading a portion of the platform will cause it to raise or lower, as shown in FIG. 22B. Such adjustable platform can be completely separate from the stem prior to assembly in-situ, or just prior to implantation, and could be another modular component of the shoulder. Such platform could be in a variety of sizes, and could be in a variety of angles, as well as offsets, for example, the head component need not be aligned with the long axis of the stem. These modular platforms could be press-fit into a corresponding cavity in the stem, or could be threaded into the stem, or pinned to the stem.

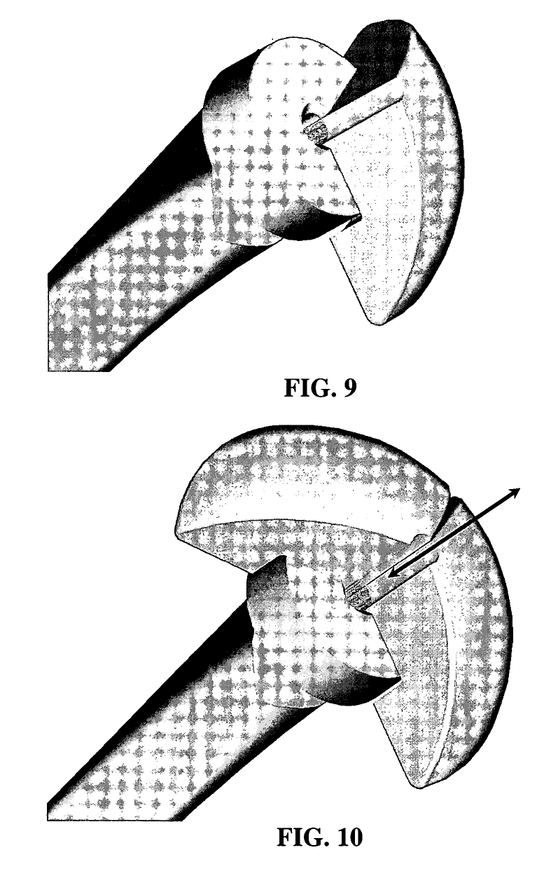

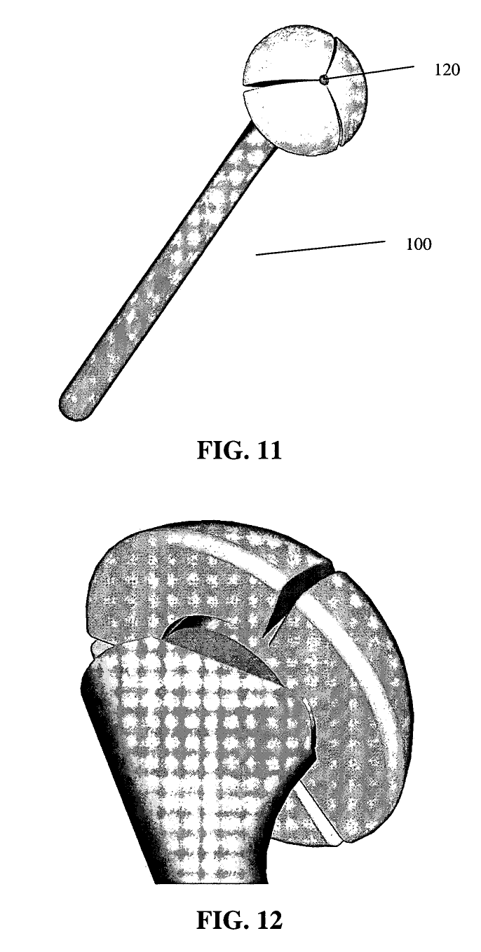

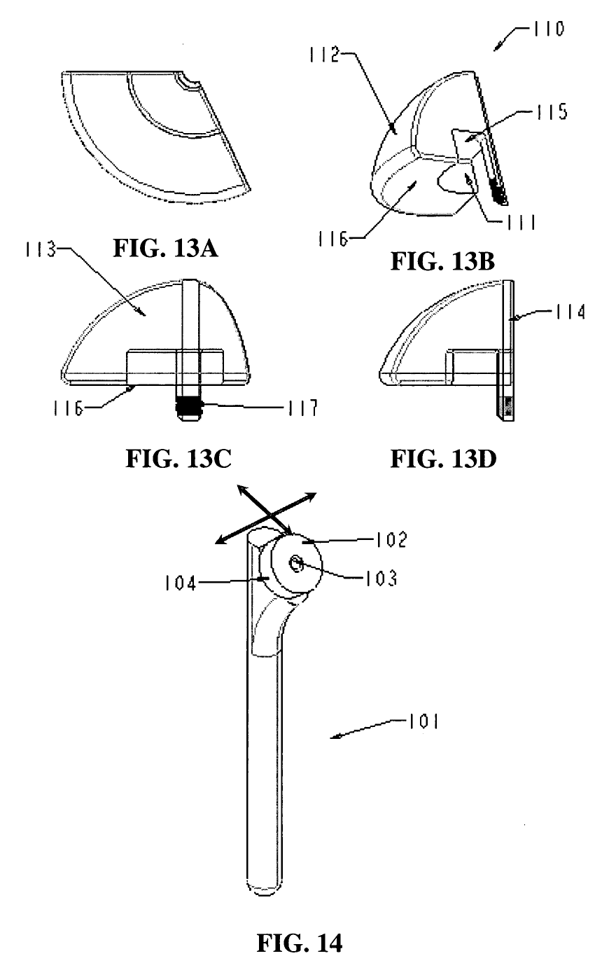

Shoulder-100 is similar to the previous embodiments, in that it contains a multi-piece head component 110, assembled in-situ. Implant 100 has head components that engage platform 104 in a manner slightly different than previously described. Head components 110 are seated through a single opening 103 in platform 104 of stem 101, as shown in FIGS. 9-14. Additionally, once all the head components are assembled, a screw, pin or other fastening means 120 is placed into opening 103, and between grooves 114 in head components, as shown in FIG. 11, thereby locking the head components onto the platform.

Variations of this design include not only having adjustable platforms as described previously, but to have head components that have a variety of geometries defining cavity 111. For example, the depth of such cavity may vary, resulting in various offsets for the head when assembled, as shown in FIG. 10, with the double arrow showing the direction of changing various offsets of the modular head components as a result of varying the depth of cavity 111. Additionally, or in combination with such cavity depth variation, such cavity, can be angled so that various assembled head angles can be created. Another embodiment would be to have the cavities offset from the center such that the head components, when assembled, would be offset in a direction perpendicular to such fastening means, as shown in FIG. 14.

A further embodiment would have an actuating rod (not shown) passing through the middle humeral head component, and intersecting with the channel in the middle component that currently houses the rod. The actuating rod would engage rod; by actuating the actuating rod, the locking and alignment rod would engage the assembled side pieces and lock them in place. Actuation would be accomplished via threading actuating rod into locking rod, and having thread or gear engagement between the two. Actuation could be accomplished via threading the actuating rod into a two-piece locking rod and wedging the two piece rod apart and into the side components, to lock and align such side components.

Alternative embodiments could include multiple locking means engaging single head components, or multiple head components simultaneously. Alternative embodiment would include multiple channels in the center piece engaging multiple channels in the side components, to either single side components, or multiple side components simultaneously.

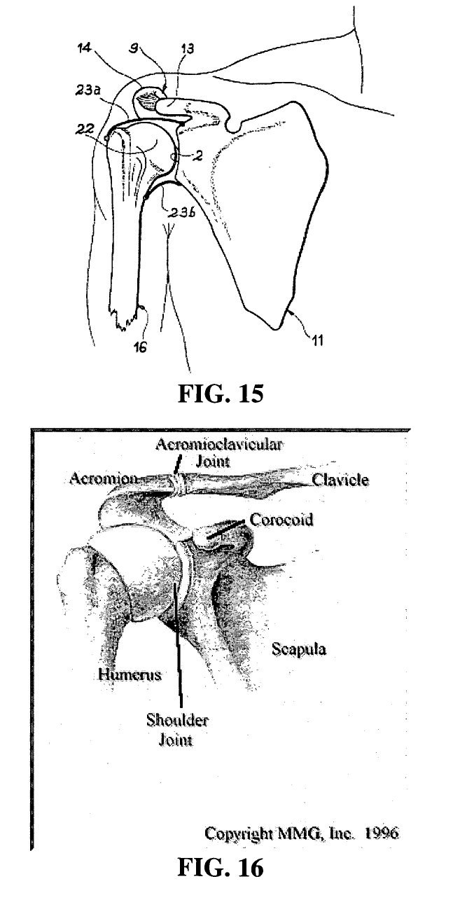

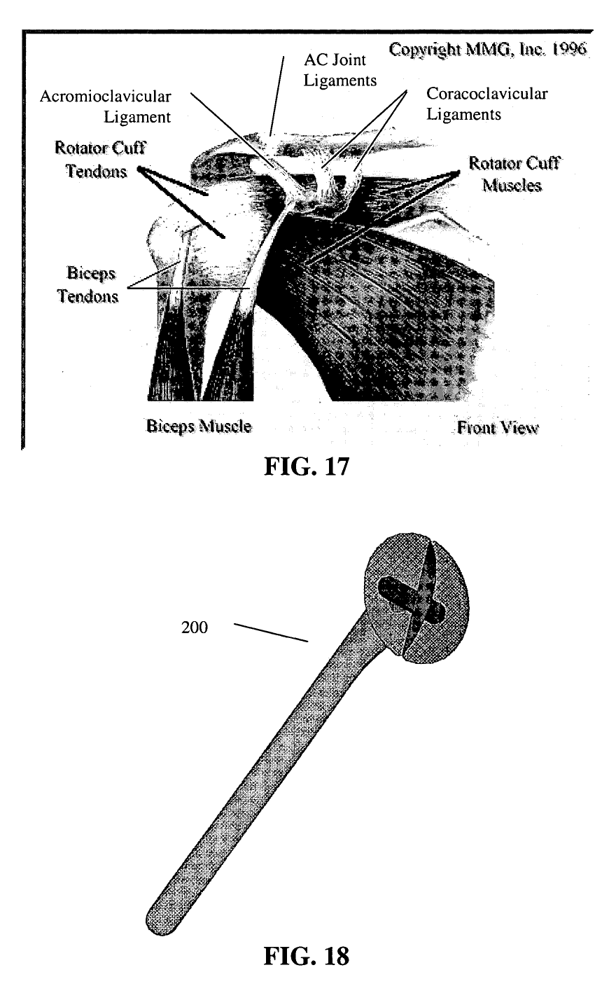

FIG. 15 shows a typical shoulder joint, taken from U.S. Pat. No. 4,693,723. Humeral head 22 articulates glenoid surface 2, and is generally constrained by soft tissues 23a and 23b. Humeral shaft 16 would house the humeral shaft of a shoulder prosthesis.

To provide more options for the surgeon to recreate the anatomy and function of the effected shoulder joint, it is advantageous for the surgeon to be able to adjust the neck height, neck length, and humeral head offset of the humeral head component. Adjustment of these variables can be accomplished in a variety of ways, such by changing the angle of the head component with respect to the stem component. Alternatively, all any combination of these variables can be adjusted, not necessary all three simultaneously, or one at a time. Embodiments that would allow for such variability to be adjusted include a series of platforms that may have a variety of offsets, thickness, angles, or a combination thereof. Such platforms are also used to link the head components with the stem components.

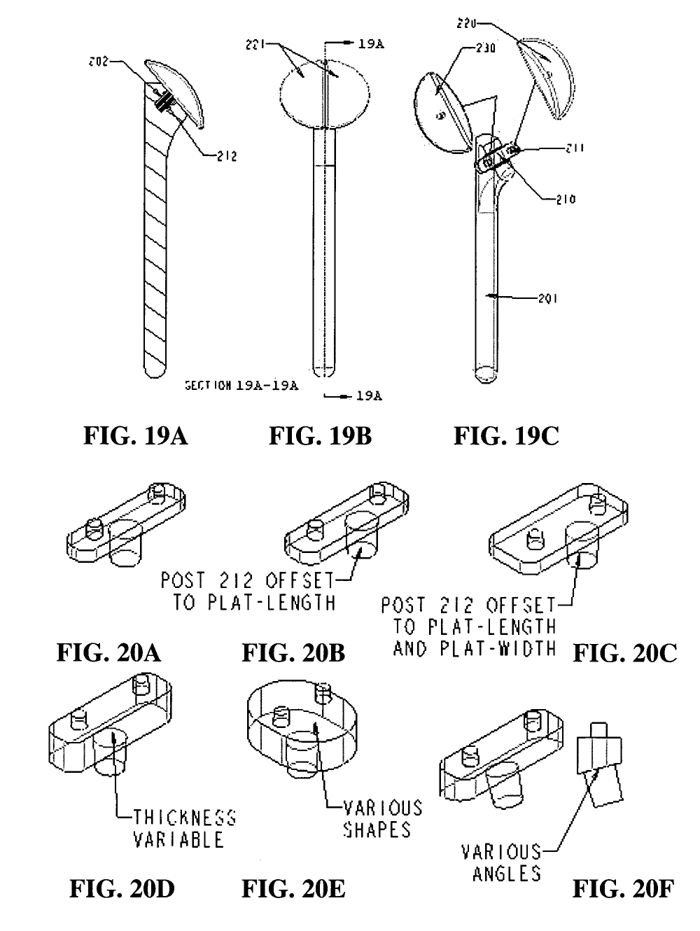

Shoulder-200 shows an alternative embodiment of the current invention, where the humeral component is comprised of a stem portion 201, to which a platform 211 is attached, as shown in FIGS. 18-19C. The humeral head components 220 and 230 are then mounted to the platform. This assembly is performed in-situ, alternatively, the surgeon may elect to complete some of the assembly prior to implantation. Platforms 211 can be of a variety of sizes to best accomadate the unique anatomy of a specific patient, and to best restore function. Examples of such a variety are shown in FIGS. 20A-20F. Although Shoulder-200 is shown with a two-piece head component, more than two pieces are envisioned. In addition, although Shoulder-200 shows the head component divided in a generally superior/inferior direction, when viewed in-vivo, such division could be anterior/posterior, lateral, or some intermediate orientation, or a combination thereof. FIGS. 18-20F also show the platform having protrusions on both the inferior surface to engage the stem, as well as on the superior portion to engage the head components. It is understood that stem and/or head components could have either protrusions or cavities to accept such protrusions.

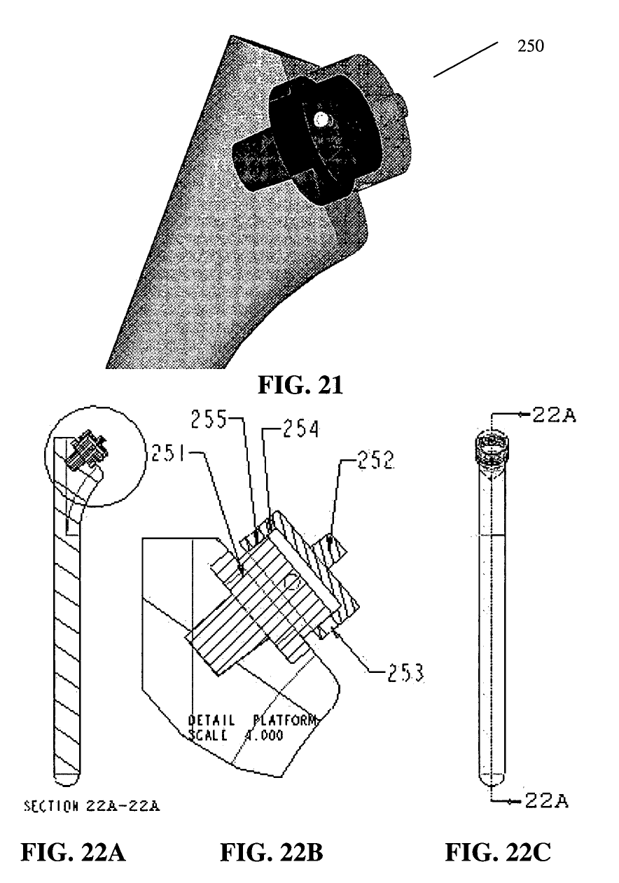

A further alternative embodiment would have a platform, either integral to the stem or head components, or a separate component that is assembled to the construct during surgery. FIGS. 21-22C show an adjustable platform 250 which is capable to adjust the relative positions between components 251 (attached to stem) and 252 (which attaches to the head). In FIG. 22B, components 252 and 251 are threadingly engaged. These two components could be adjusted through a hole 253, generally perpendicular to the long axis of platform 250. Alternatively, adjustability could be performed through a hole (not shown) through the protrusion engaging head components. Once the height has been appropriately adjusted, this height can be locked in place through at least one set screw threaded through hole(s) 253. Alternatively, a threaded nut (not shown) could be threaded along threads 255 of component 251, until it engages and frictionally locks to the lower surface of component 252. Alternatively, such adjustable platform can be rotatably adjustable, to allow adjustability with respect to eccentricity of the head component, but not necessary neck length adjustable. For example, the rotatably adjustable means for adjusting eccentricity simply rotates, it does not expand.

Actuation means can include, but are not limited to, threaded gears, compressed air, fluid, wedging, hardenable resins. Such threaded gears could have side pieces internally attached to a threaded rod, and such rod engaged with an actuation rod which would be generally perpendicular to such threaded rod, and contained within the center component. Such actuation rod will have means to actuate it from outside the cavity of the center component. Such medium could be contained within an internal bladder. Wedging could aid in expansion of the side components by having such wedges attached internally to the side pieces, and having an actuation rod in the central component that will force the wedges apart, thereby expanding the head. Expansion will be completed when the side components have reached their final position. Such final position can be defined by stops on internal guide rails, stops on outer edge of central component, to engage with corresponding stops on outer edges of side components.

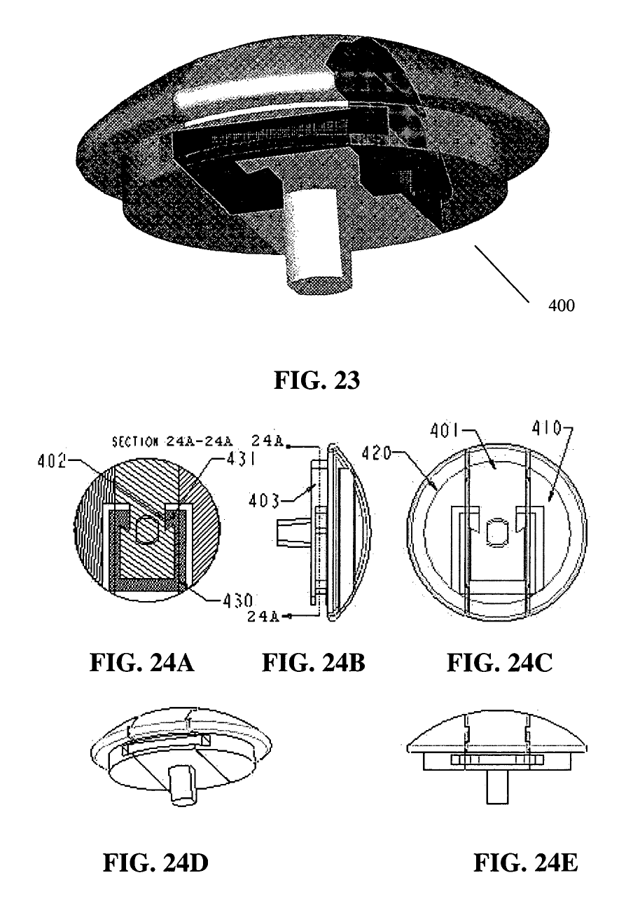

A further embodiment of a modular head component is illustrated in FIGS. 23-24E as being modular head assembly 400, which is composed of, as shown but not limited to, three components 401, 410, and 420, which make-up the articulating head portion. Additionally, retaining clip 430 is inserted into a cavity shared between at least the outer head components, but shown to be shared between all three components. Such retaining clip is inserted into such cavity, and leading prongs engage an appropriately contoured cavity, so that the clip retains all three components, by compression of the components between the inner surfaces of the retaining clip. The fishhook-like tips of the clip prevent the clip from backing out of such cavities. Such cavity and clip can be located anywhere on the head components.

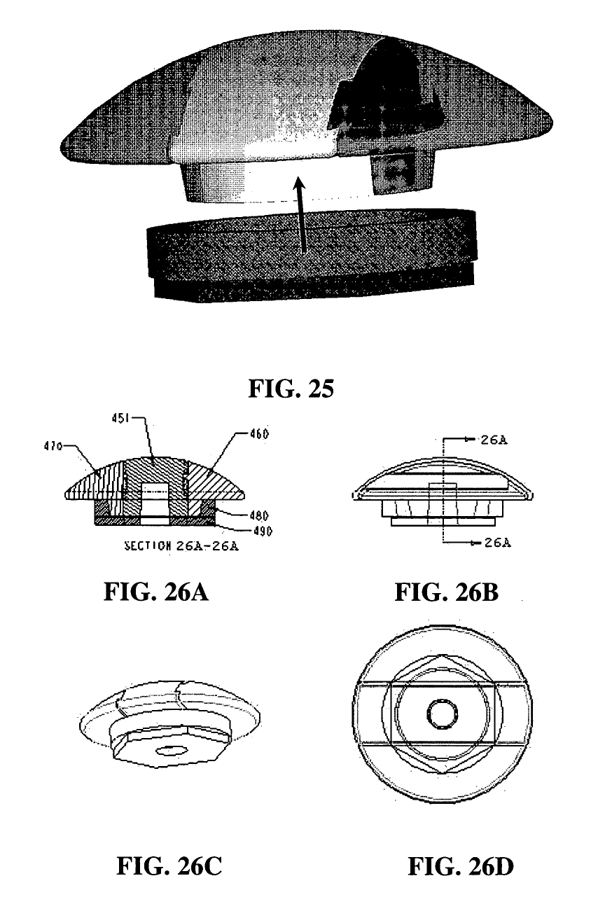

A further embodiment of a modular head is illustrated in FIGS. 25-26D. Such modular head assembly 450 is composed of, as shown but not limited to, three components 451, 460, and 470, which make-up the articulating head portion. Elements 480 and 490 form a retaining ring sub-assembly that may be attached to the stem component of the implant. Element 490 is a ring that matingly engages a protrusion (composed from at least two of the head components, as shown, this is three head components) on the distal surface of the head components, via a taper lock. The stem component has a protrusion that will matingly engage with the head component assembly, and will pass through the retaining ring subassembly. Elements 480 and 490 can be threadingly engaged with each other, to allow, for example, element 480 to be threaded onto a corresponding portion of the stem.

A further embodiment of the invention is a guide instrument that will aid in the in-situ assembly of the modular head of the prosthesis system. Such guide (not shown) could be generally of a tuning-fork shape, with each of the two prongs engaging the cavities. The edges of the prongs that are not engaging the such cavities, will extend slightly past the trailing end of the modular head component, and act as a guide rail for the side components to translate along, slip into, and engage the such cavities. Male protrusions on side components of the modular head component would have a groove on the surface facing cavities. Such groove will be that which slides along the guide rails. In this manner, the guide rail will grip a length of cavities, but will not have a height that will engage the height of such cavity, and will allow the side components to translate along the guide rails, and easily allow assembly of the side components with the center component. After the assembly is complete, the guide rail will be removed.

It is understood that the inventions and features disclosed herein are not limiting, and that features discussed in one embodiment may easily be applied to others. The ideas described herein can be interchangeable with the various embodiments described. For example, features of embodiments that are assembled perpendicular to the long axis of the stem humeral stem can be applied to embodiments that are assembled more parallel to the long axis of the humeral stem.

What is claimed is:

1. A joint prosthesis, comprising a head having multiple components for assembly with each other in-situ, each having an outward surface that cooperates with each other to enable articulation against an external surface, each having a formation configured to cooperate with each other to effect mechanical connection that retains the multiple components in position relative to each other.

2. The joint prosthesis of claim 1, wherein the outward surfaces of the head are configured to articulate with a natural glenoid gravity.

3. The joint prosthesis of claim 1, further comprising a stem having proximal and distal ends, the distal end having an elongated stem portion configured to fit within a canal of a bone, and the proximal end having a different shape than that of the distal end.

4. The joint prosthesis of claim 3, wherein a portion of the distal of the stem has a surface texture suited to facilitate the fitting of the stem in the canal.

5. The joint prosthesis of claim 3, wherein the formation of at least one of the components has a mating configuration to mate with a complementary mating configuration at the proximal end of the stem.

6. The joint prosthesis of claim 3, further comprising at least one platform positioned between the head and the stem, wherein the platform is configured to matingly engage the head and the stem.

7. The joint prosthesis of claim 6, wherein the platform is configured to be adjustable to change a distance between the head component and the stem component.

8. The joint prosthesis of claim 6, wherein the platform being configured to be rotatable to adjust a position of the head component relative to the stem component.

9. The joint prosthesis of claim 1, wherein said head has at least one protrusion that is configured to engage a periphery of at least one corresponding cavity so as to block the protrusion from rotating within said corresponding cavity.

10. The joint prosthesis of claim 1, wherein said head has multiple protrusions that are configured to fit simultaneously into one single cavity, the cavity having a periphery configured to block rotation of the multiple mechanisms within the cavity.

11. The joint prosthesis of claim 9, wherein said at least one protrusion constitutes multiple protrusions, and at least one cavity constitutes multiple cavities, each of the multiple protrusions being configured to fit into a corresponding one of the multiple cavities.

12. The joint prosthesis of claim 9, further comprising a securing means for retaining and leveling said multiple components into said cavity.

13. The joint prosthesis of claim 12, wherein the securing means is selected from a group consisting of a screw, a press-fit rod, and a rivet.

14. The joint prosthesis of claim 1, wherein the outward facing surface of each of the multiple components represents a portion of a continuous and smooth surface to ensure continuous and smooth articulation.

15. The joint prosthesis of claim 14, wherein the outward surface of each of the multiple components together form a shape that is substantially hemispherical.

16. The joint prosthesis of claim 15, wherein the multiple components represent sections of said shape divided radially from each other.

17. The joint prosthesis of claim 16, further comprising gaps between every two adjacent sections.

18. The joint prosthesis of claim 1, further comprising a retainer to retain the components in place relative to each other by engaging the formations.

19. The joint prosthesis of claim 18, wherein the formations include at least one cavity in which the retainer is inserted.

20. The joint prosthesis of claim 18, wherein the formations include at least one peripheral configuration engaged by the retainer.

21. A method of assembling a joint prosthesis, comprising assembling multiple components of a head together wherein each component has an outward facing surface that cooperates with each other to enable articulation against an external surface, each having a formation configured to effect mechanical connection to retain an associated one of the multiple components relative to other ones of the multiple components whose formations are mechanically connected.

22. The assembling method of claim 21, wherein the assembling is at least partially carried out in-situ in a patient receiving an implantation of the joint prosthesis.

23. A method of articulating a joint prosthesis, comprising assembling multiple components of a head together wherein the multiple components being configured in that each component has an outward facing surface that cooperates with each other to enable articulation against an external surface, and articulating the multiple outward facing surfaces against the external surface, each having a formation configured to effect mechanical connection to retain an associated one of the multiple components relative to other ones of the multiple components whose formations are mechanically connected.
